(19) United States
(12) Patent Application Publication
(10) Pub. No.: US 2025/0241510 A1
(43) Pub. Date: Jul. 31, 2025

(54) PROCESSING DEVICE, ENDOSCOPE SYSTEM, DETECTION VALUE CALCULATION METHOD AND COMPUTER-READABLE RECORDING MEDIUM

(71) Applicant: OLYMPUS MEDICAL SYSTEMS CORP., Tokyo (JP)

(72) Inventor: Ryuichi YAMAZAKI, Tokyo (JP)

(73) Assignee: OLYMPUS MEDICAL SYSTEMS CORP., Tokyo (JP)

(21) Appl. No.: 19/041,229

(22) Filed: Jan. 30, 2025

Related U.S. Application Data
(60) Provisional application No. 63/627,186, filed on Jan. 31, 2024.

(51) Int. Cl. A61B 1/00; A61B 1/04; A61B 1/06; A61B 34/20; G06V 10/14; G06V 10/25; G06V 10/60; G06V 10/764; G16H 30/40; H04N 23/50; H04N 23/56; H04N 23/71; H04N 23/76

(52) U.S. Cl. CPC A61B 1/00009; A61B 34/20; G06V 10/14; G06V 10/25; G06V 10/60; G06V 10/764; G16H 30/40; H04N 23/555; H04N 23/56; H04N 23/71; H04N 23/76; A61B 1/04; A61B 1/0661; A61B 2034/2065; G06V 2201/034

(57) ABSTRACT

A processing device includes: one or more processors including hardware, the one or more processors being configured to receive an observation image captured by an endoscope, calculate a representative luminance value of each of plural luminance areas resulting from classification of a specific detection area in an observation image according to luminance values, determine a treatment tool area in the observation image, count detected pixels exceeding a specific luminance value from pixels in the detection area, reduce a counted number of the detected pixels in the treatment tool area from a counted number of the detected pixels of the pixels in the detection area, calculate a weight of each of the plural luminance areas, and adjust brightness of the observation image based on the weight and the representative luminance value of each of the plural luminance areas.

### CROSS-REFERENCE TO RELATED APPLICATION

This application is based on and claims priority under 35 U.S.C. § 119 to U.S. Provisional Application No. 63/627,186, filed Jan. 31, 2024, the entire contents of which are incorporated herein by reference.

### BACKGROUND

### 1. Technical Field

The present disclosure relates to a detection value calculation device, an endoscope system, a detection value calculation method, and a computer-readable recording medium.

### 2. Related Art

In a known endoscope system for observation of a subject, such as the interior of a living body, the subject is irradiated with illumination light by use of a light source device, and returned light (a subject image) from the subject is captured by use of an endoscope (see, for example, Japanese Patent No. 4223778).

In the endoscope system described in Japanese Patent No. 4223778, a detection value (overall luminance value) for adjusting brightness of an observation image captured by use of the endoscope is calculated from luminance values of the observation image, and adjustment of light quantity of the illumination light for the subject (light adjustment control) at the light source device is performed on the basis of the detection value.

In a case where the endoscope system is used in, for example, surgery, a treatment tool, such as forceps, may be included in the observation image. In this case, reflection of the illumination light by a surface of the treatment tool, such as forceps, causes halation in a portion of the treatment tool in the observation image, and the detection value is unable to be calculated adequately. That is, executing the light adjustment control on the basis of this detection value reduces the illumination light so that the halation is minimized, and thus darkens a region of interest that a user, such as a medical doctor, wishes to observe, not resulting in obtainment of an observation image suitable for observation.

FIG. 9 is a diagram for description of a problem to be solved by the disclosed embodiments. Specifically, FIG. 9 is a diagram illustrating transitions of detection values and average luminance values of the whole observation image when light quantity of illumination light is gradually increased. In FIG. 9, the horizontal axis corresponds to time and the vertical axis to the detection value and average luminance value. Furthermore, a line L1 in FIG. 1 represents the detection value. A line L2 represents the average luminance value of the whole observation image.

In the endoscope system described in Japanese Patent No. 4223778, in a case where a luminance value of an area of a treatment tool expected to be captured in the observation image is larger than a boundary luminance value, the detection value is calculated from luminance values of an area other than the area of the treatment tool in the observation image. Therefore, a large change in the detection value is generated around the boundary luminance value, resulting in a discontinuous transition (see a transition T in FIG. 9) in the average luminance value of the whole observation image and not resulting in obtainment of an observation image suitable for observation.

Therefore, there is a demand for a technique enabling a detection value to be calculated adequately even in a case where a treatment tool, such as forceps, is included in an observation image.

### SUMMARY

In some embodiments, a processing device includes: one or more processors comprising hardware, the one or more processors being configured to receive an observation image captured by an endoscope, calculate a representative luminance value of each of plural luminance areas resulting from classification of a specific detection area in an observation image according to luminance values, determine a treatment tool area in the observation image, count detected pixels exceeding a specific luminance value from pixels in the detection area, reduce a counted number of the detected pixels in the treatment tool area from a counted number of the detected pixels of the pixels in the detection area, calculate a weight of each of the plural luminance areas based on the counted number of the detected pixels subjected to the reducing, and adjusting brightness of the observation image based on the weight and the representative luminance value of each of the plural luminance areas.

In some embodiments, provided is a method executed by a processor of a detection value calculation device. The method includes: receiving an observation image captured by an endoscope,
calculating a representative luminance value of each of plural luminance areas resulting from classification of a specific detection area in an observation image according to luminance values, determining a treatment tool area in the observation image, counting detected pixels exceeding a specific luminance value from pixels in the detection area, executing adjustment processing of reducing a counted number of the detected pixels in the treatment tool area from a counted number of the detected pixels of the pixels in the detection area, calculating a weight of each of the plural luminance areas based on the counted number of the detected pixels subjected to the reducing, and adjusting brightness of the observation image based on the weight and the representative luminance value of each of the plural luminance areas.

In some embodiments, provided is a non-transitory computer-readable recording medium with an executable program stored thereon. The program causes a processor to execute: receiving an observation image captured by an endoscope, calculating a representative luminance value of each of plural luminance areas resulting from classification of a specific detection area in an observation image according to luminance values, determining a treatment tool area in the observation image, counting detected pixels exceeding a specific luminance value from pixels in the detection area, reducing a counted number of the detected pixels in the treatment tool area from a counted number of the detected pixels of the pixels in the detection area, calculating a weight of each of the plural luminance areas based on the counted number of the detected pixels that has been subjected to the adjustment processing, and adjusting brightness of the observation image based on the weight and the representative luminance value of each of the plural luminance areas.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration of an endoscope system according to an embodiment;

FIG. 2 is a flowchart illustrating operation of a processor;

FIG. 3 is a diagram for description of the operation of the processor;

FIG. 4 is a diagram for description of the operation of the processor;

FIG. 5 is a diagram for description of a first modified example of the embodiment;

FIG. 6 is a diagram for description of the first modified example of the embodiment;

FIG. 7 is a diagram for description of a second modified example of the embodiment;

FIG. 8 is a diagram for description of a third modified example of the embodiment; and

FIG. 9 is a diagram for description of a problem to be solved by the disclosed embodiments.

### DETAILED DESCRIPTION

A mode for implementing the disclosed embodiments will be described hereinafter while reference is made to the drawings. The present invention is not to be limited by the embodiment described hereinafter. The same reference sign will be assigned to portions that are the same in the drawings.

### Configuration of Endoscope System

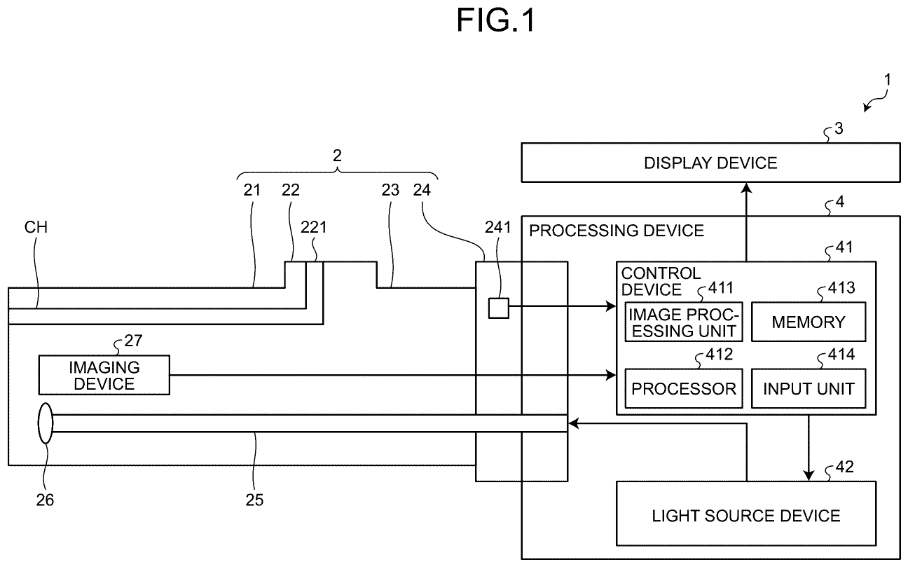

FIG. 1 is a diagram illustrating a configuration of an endoscope system 1 according to an embodiment.

The endoscope system 1 is an endoscope system that is used in the medical field and that is for observation of the interior of a subject (inside a living body) by use of an endoscope. As illustrated in FIG. 1, this endoscope system 1 includes an endoscope 2, a display device 3, and a processing device 4.

In this embodiment, the endoscope 2 is a so-called flexible endoscope. The endoscope 2 is not necessarily a flexible endoscope, and a so-called rigid endoscope may be adopted as the endoscope 2 instead. Part of the endoscope 2 is inserted into a living body, and the endoscope 2 captures images of the interior of the living body and outputs image signals generated by the capturing of the images. The endoscope 2 includes, as illustrated in FIG. 1, an insertion unit 21, an operating unit 22, a universal cord 23, and a connector unit 24.

At least part of the insertion unit 21 has flexibility and the insertion unit 21 is a portion to be inserted into the living body. A light guide 25, an illumination lens 26, and an imaging device 27 have been provided in the insertion unit 21.

The light guide 25 is led to the connector unit 24 from the insertion unit 21 through the operating unit 22 and the universal cord 23. One end of the light guide 25 is positioned in a distal end portion of the insertion unit 21. Furthermore, in a state where the endoscope 2 has been connected to the processing device 4, the other end of the light guide 25 is positioned in the processing device 4. The light guide 25 transmits light supplied from a light source device 42 in the processing device 4 from the other end to the one end.

The illumination lens 26 faces the one end of the light guide 25 in the insertion unit 21. The illumination lens 26 emits light transmitted by the light guide 25 to the interior of the living body.

The imaging device 27 is provided in the distal end portion of the insertion unit 21. The imaging device 27 captures images of the interior of the living body and outputs image signals generated by the capturing of the images. Specific illustration of the imaging device 27 has been omitted, but the imaging device 27 includes an imaging optical system and an imaging unit.

The imaging optical system captures light (a subject image) that has been emitted to the interior of the living body from the illumination lens 26 and returned from the interior of the living body and forms an image on an imaging surface of an imaging element included in the imaging unit.

The imaging unit is configured to include the imaging element, such as a charge coupled device (CCD) or a complementary metal oxide semiconductor (CMOS), captures the subject image formed by the imaging optical system, and outputs an image signal generated by the capturing.

The image signal generated by the imaging device 27 will hereinafter be referred to as an observation image, for convenience of description.

The operating unit 22 is connected to a proximal end portion of the insertion unit 21. The operating unit 22 receives various kinds of operation for the endoscope 2. Furthermore, the operating unit 22 has an insertion opening 221 provided therein, as illustrated in FIG. 1. This insertion opening 221 is an insertion opening communicating with a treatment tool channel CH (pipe conduit) extending from a distal end of the insertion unit 21 and is for inserting a treatment tool, such as forceps (not illustrated in the drawings), from outside into the treatment tool channel CH.

From the operating unit 22, the universal cord 23 extends in a direction different from a direction, in which the insertion unit 21 extends, and the universal cord 23 is a cord having a signal line and a light guide 25, for example, arranged therein, the signal line being for electrically connecting the imaging device 27 and a control device 41 in the processing device 4.

The connector unit 24 is provided at an end portion of the universal cord 23 and is detachably connected to the processing device 4. The connector unit 24 has a memory 241 provided therein. This memory 241 has treatment tool area information recorded therein. This treatment tool area information is information specific to the endoscope 2 and is information indicating an area of a treatment tool expected to be captured in an observation image from positions of the imaging optical system, the illumination lens 26, and the treatment tool channel at the distal end of the insertion unit 21 (coordinate values of pixels in the area of the treatment tool).

The display device 3 is, for example, a liquid crystal display (LCD) or an electroluminescence (EL) display, and displays, for example, an observation image resulting from execution of image processing by the processing device 4.

The processing device 4 includes, as illustrated in FIG. 1, the control device 41 and the light source device 42. In this embodiment, the light source device 42 and the control device 41 are provided as the processing device 4 in a single housing but without being limited to this embodiment, the light source device 42 and the control device 41 may be respectively provided in separate housings.

The light source device 42 supplies illumination light, such as white light or excitation light that excites a fluorescent substance included in the living body, to the other end of the light guide 25, under control by the control device 41.

The control device 41 corresponds to a detection value calculation device and a light source control device. This control device 41 integrally controls the overall operation of the endoscope system 1. The control device 41 includes, as illustrated in FIG. 1, an image processing unit 411, a processor 412, a memory 413, and an input unit 414.

The image processing unit 411 executes image processing of an observation image generated by the endoscope 2, generates a video signal for display for the observation image that has been subjected to the image processing to be displayed by the display device 3, and outputs the video signal to the display device 3. Examples of the image processing executed by this image processing unit 411 include: optical black subtraction processing (clamping processing), white balance adjustment processing, demosaicing processing, color correction matrix processing, gamma correction processing, YC processing for conversion of R, G, and B signals to luminance and color difference signals (Y and Cb/Cr signals), gain adjustment, denoising, and filtering processing for structure enhancement.

The processor 412 is implemented by various programs (including a detection value calculation program) being executed by a controller, such as a central processing unit (CPU) or a microprocessing unit (MPU), the various programs having been recorded in the memory 413. The processor 412 controls operation of the endoscope 2, the light source device 42, and the display device 3, and controls the overall operation of the control device 41. The processor 412 is not necessarily a CPU or an MPU and may include an integrated circuit, such as an application specific integrated circuit (ASIC) or a field programmable gate array (FPGA).

Functions of the processor 412 will be described later in “Operation of Processor”.

The memory 413 has, recorded therein, the various programs executed by the processor 412 (including the detection value calculation program) and information needed in processing by the processor 412, for example.

The input unit 414 is configured by use of a keyboard, a mouse, a switch, and/or a touch panel, for example, and receives user operation by a user, such as an operating surgeon. The input unit 414 then outputs an operation signal corresponding to the user operation, to the processor 412.

### Operation of Processor

Operation of the above described processor 412 (including a detection value calculation method) will be described next.

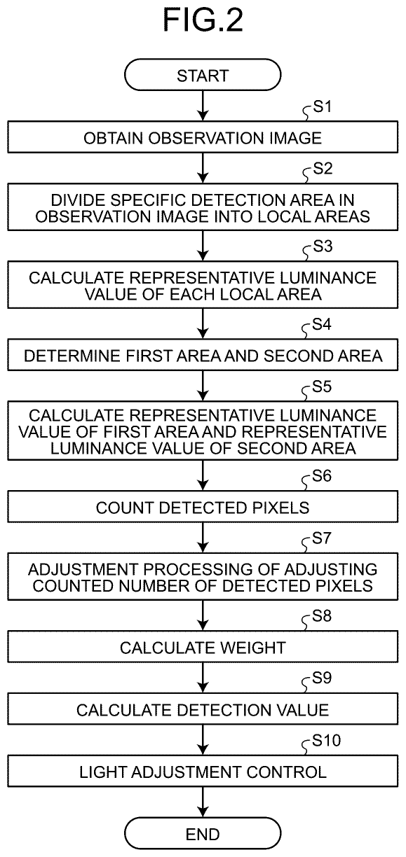

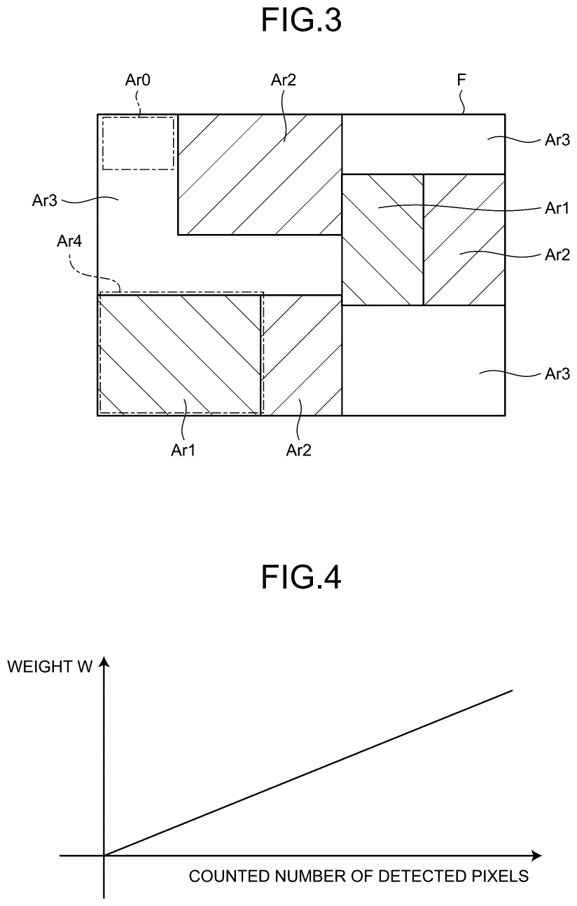

FIG. 2 is a flowchart illustrating the operation of the processor 412. FIG. 3 and FIG. 4 are diagrams for description of the operation of the processor 412. Specifically, FIG. 3 is a diagram illustrating an observation image F. FIG. 4 is a diagram illustrating an example of relation information indicating a relation between the counted number of detected pixels and weight W. In FIG. 4, the horizontal axis corresponds to the counted number of detected pixels and the vertical axis to the weight W.

Firstly, the processor 412 obtains the observation image F resulting from execution of image processing by the image processing unit 411 (Step S1).

After Step S1, the processor 412 divides a specific detection area in the observation image F obtained at Step S1 into local areas Ar0 (Step S2). For convenience of description, in FIG. 3, only one local area Ar0 is illustrated with a dash dotted line. In this embodiment, the whole area of the observation image F is adopted as the detection area. The detection area is not necessarily the whole area of the observation image F and may be an area of part of the observation image F. Furthermore, in this embodiment, local areas are areas resulting from division of the observation image F (detection area) into 25 areas of 5×5. The local areas may be areas resulting from division of the observation image F (detection area) into any other number of areas, such as nine areas of 3×3, for example.

After Step S2, the processor 412 calculates a representative luminance value for each local area Ar0 (Step S3).

An example of the representative luminance value of the local area Ar0 may be an average luminance value resulting from averaging of respective luminance values of all of pixels in the local area Ar0. The representative luminance value is not necessarily the average luminance value, and the highest luminance value, the lowest luminance value, or the median luminance value among all of the pixels in the local area Ar0 may be adopted as the representative luminance value.

After Step S3, the processor 412 determines a first area Ar1 and a second area Ar2 in the observation image F (detection area) (Step S4).

Specifically, the processor 412 determines, as the first area Ar1 (FIG. 3), all of local areas Ar0 having representative luminance values equal to or larger than a specific first luminance value. Furthermore, the processor 412 determines, as a third area Ar3 (FIG. 3), all of local areas Ar0 having representative luminance values equal to or less than a second luminance value smaller than the first luminance value. Furthermore, the processor 412 determines, as the second area Ar2 (FIG. 3), all of local areas Ar0 having representative luminance values smaller than the first luminance value and larger than the second luminance value. That is, at Step S4, the processor 412 classifies the observation image F (detection aera) into plural luminance areas (the first area Ar1, the second area Ar2, and the third area Ar3) according to the luminance values.

After Step S4, the processor 412 calculates each of a representative luminance value of the first area Ar1 and a representative luminance value of the second area Ar2 (Step S5).

An example of the representative luminance value of the first area Ar1 may be an average luminance value resulting from averaging of respective representative luminance values of all of the local areas Ar0 included in the first area Ar1. This representative luminance value is not necessarily the average luminance value, and the highest representative luminance value, the lowest representative luminance value, or the median representative luminance value, of all of the local areas Ar0 included in the first area Ar1 may be adopted as the representative luminance value. The representative luminance value of the second area Ar2 is similar to the representative luminance value of the first area Ar1.

After Step S5, the processor 412 counts detected pixels of all of pixels in the observation image F, the detected pixels having luminance values equal to or larger than a specific luminance value (Step S6).

After Step S6, the processor 412 executes adjustment processing of adjusting a counted number of the detected pixels counted at Step S6 (Step S7).

Specifically, the processor 412 obtains the treatment tool area information from the memory 241 of the endoscope 2. Furthermore, on the basis of the treatment tool area information, the processor 412 determines a treatment tool area Ar4 (FIG. 3). Furthermore, the processor 412 reads reflection rate information indicating a reflection rate from the memory 413. The reflection rate is a value equal to or larger than 0 and smaller than 1. The processor 412 then executes the adjustment processing of reducing the counted number of the detected pixels counted at Step S6 by performing a multiplication by the reflection rate according to a counted number of detected pixels in the treatment tool area Ar4 among the counted number of the detected pixels counted at Step S6. For example, in a case where the reflection rate is 0, the counted number of the detected pixels in the treatment tool area Ar4 is 0, and the counted number of the detected pixels counted at Step S6 is thus determined as a counted number of detected pixels outside the treatment tool area Ar4.

After Step S7, the processor 412 calculates weight W on the basis of the counted number of the detected pixels that has been subjected to the adjustment processing (Step S8).

Specifically, the processor 412 reads relation information from the memory 413. In this embodiment, the relation information is information indicating a relation, in which the weight W linearly increases as the counted number of the detected pixels increases, as illustrated in FIG. 4. The processor 412 then calculates weight W corresponding to the counted number of the detected pixels that has been subjected to the adjustment processing, on the basis of the relation information.

After Step S8, the processor 412 calculates a detection value for adjusting brightness of the observation image F from the following Equation 1, on the basis of the weight W and the representative luminance values of the first area Ar1 and second area Ar2 (Step S9).

$$Detection value = W \times ( representative luminance value of first area Ar 1 ) + ( 1 - W ) \times ( representative luminance value of second area Ar 1 ) \qquad ( 1 )$$

After Step S9, the processor 412 executes light adjustment control of the light source device 42 (Step S10).

Specifically, the processor 412 executes the light adjustment control of adjusting light quantity of illumination light supplied from the light source device 42 so that the detection value calculated at Step S9 will be at a luminance value targeted.

The above described embodiment has the following effects.

The processor 412 in the control device 41 according to this embodiment executes the above described processing at Steps S1 to S9. Therefore, in a case where a treatment tool, such as forceps, has been captured in the observation image F, the weight W is able to be decreased and relative importance of the first area Ar1 in calculation of a detection value is able to be reduced. On the contrary, in a case where a treatment tool, such as forceps, has not been captured in the observation image F, the weight W is increased and the relative importance of the first area Ar1 in calculation of a detection value is not reduced.

Therefore, the control device 41 according to this embodiment enables a detection value to be calculated adequately even in a case where a treatment tool, such as forceps, is included in the observation image F. As a result, light adjustment control based on the detection value enables a region of interest to have adequate brightness and the observation image F to be suitable for observation, the region of interest being where a user, such as a medical doctor, wishes to observe.

### OTHER EMBODIMENTS

A mode for implementing the disclosed embodiments has been described thus far, but the present invention is not to be limited only to the embodiments described above.

The following first to fifth modified examples may be adopted in the embodiment.

### First Modified Example

In the above described embodiment, two luminance areas, the first area Ar1 and the second area Ar2, are adopted as the plural luminance areas in calculating a detection value, but without being limited to this embodiment, three or more luminance areas may be adopted. For example, in calculating a detection value, three luminance areas, the first area Ar1, the second area Ar2, and the third area Ar3, may be adopted. In this case, at Step S9, the processor 412 calculates a detection value from the following Equation 2.

$$Detection value = W 1 \times ( representative luminance value of first area Ar 1 ) + W 2 \times ( representative luminance value of second area Ar 2 ) + ( 1 - W 1 + W 2 ) \times ( representative luminance value of third area Ar 3 ) \qquad ( 2 )$$

In Equation 2, the representative luminance value of the third area Ar3 is calculated similarly to the representative luminance values of the first area Ar1 and second area Ar2.

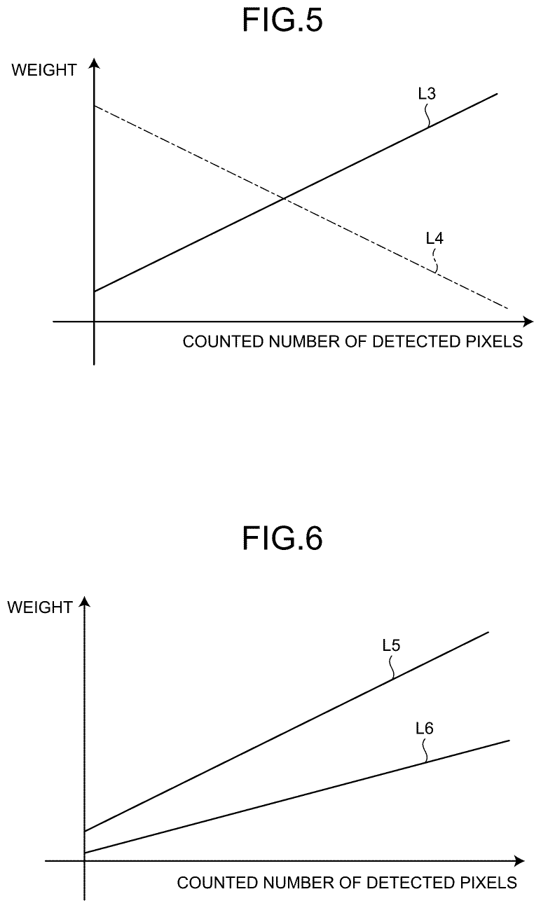

FIG. 5 and FIG. 6 are diagrams for description of the first modified example of the embodiment.

Specifically, FIG. 5 and FIG. 6 are diagrams illustrating examples of relation information indicating a relation between the counted number of the detected pixels and weight. In FIG. 5 and FIG. 6, the horizontal axis corresponds to the counted number of detected pixels and the vertical axis to weight W.

In the example of FIG. 5, the relation information includes information indicating a relation in which weight W1 linearly increases as the counted number of detected pixels increases as represented by a line L3, and information indicating a relation in which weight W2 linearly decreases as the counted number of detected pixels increases as represented by a line L4. The processor 412 calculates the weight W1 and the weight W2 corresponding to the counted number of detected pixels that has been subjected to the adjustment processing, on the basis of the relation information, and calculates a detection value from Equation 2.

In the example of FIG. 6, the relation information includes information indicating a relation in which weight W1 linearly increases as the counted number of detected pixels increases as represented by a line L5, and information indicating a relation in which weight W2 linearly increases as the counted number of detected pixels increases as represented by a line L6. The processor 412 calculates the weight W1 and the weight W2 corresponding to the counted number of detected pixels that has been subjected to the adjustment processing, on the basis of the relation information, and calculates a detection value from Equation 2.

In a case where three or more luminance areas are adopted as the plural luminance areas, like in the first modified example described above, effects similar to those of the embodiment described above are also achieved.

### Second Modified Example

In a configuration that may be adopted in the above described embodiment, Step S7 is executed only in a case where whether or not a treatment tool, such as forceps, has been captured in the observation image F is determined and it is determined that the treatment tool has been captured therein (and Step S8 is executed after Step S6 in a case where it is determined that the treatment tool has not been captured therein).

The following first to third determination methods may be adopted as methods of determining whether or not a treatment tool, such as forceps, has been captured in the observation image F.

In the first determination method, the processor 412 determines whether or not a treatment tool, such as forceps, has been captured in the observation image F by pattern matching, which is a publicly known technique.

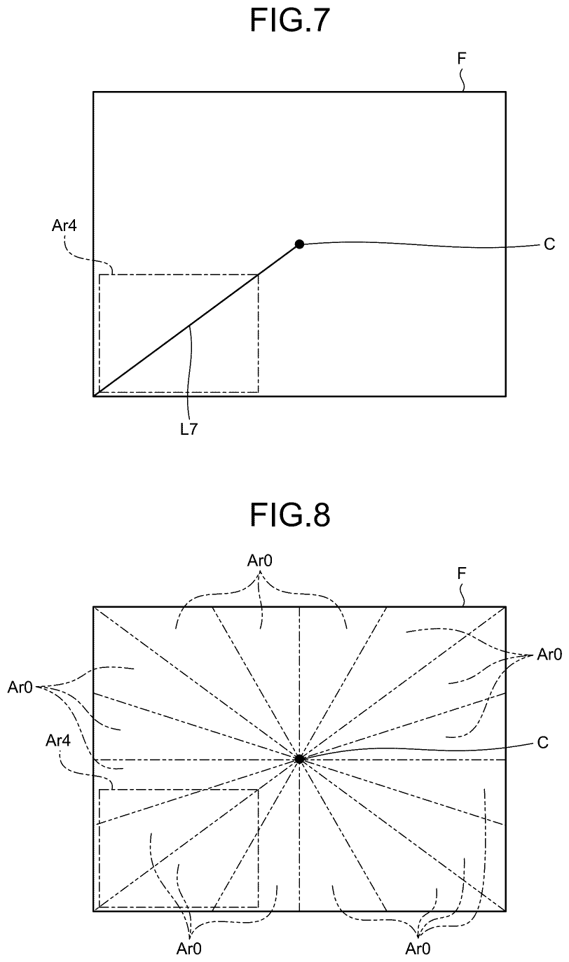

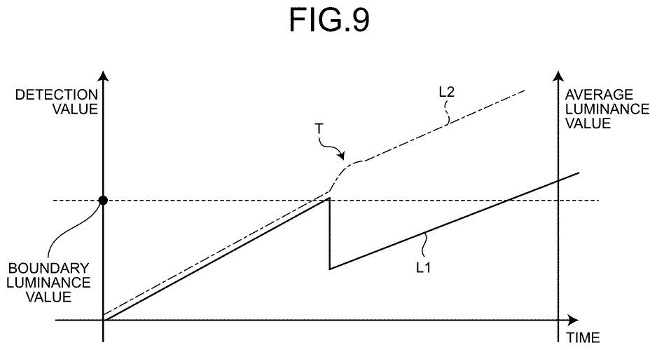

FIG. 7 is a diagram for description of the second modified example of the embodiment. Specifically, FIG. 7 is a diagram illustrating the observation image F.

In a case where a treatment tool, such as forceps, has been captured in the observation image F, a gradient (luminance distribution) of luminance values on a line L7 directed to a center position C of the observation image F from a corner of the observation image F in the treatment tool area Ar4 represents a specific gradient. In the second determination method, the processor 412 determines that a treatment tool, such as forceps, has been captured in the observation image F in a case where the gradient of the luminance values on the line L7 represents the specific gradient, and determines that the treatment tool has not been captured in the observation image F in a case where the gradient does not represent the specific gradient.

In the third determination method, the processor 412 determines that a treatment tool, such as forceps, has been captured in the observation image F in a case where a specific switch provided in the operating unit 22 of the endoscope 2 has been pressed down, and determines that the treatment tool has not been captured in the observation image F in a case where the specific switch has not been pressed down.

In a case where the above described configuration of the second modified example is adopted, Step S7 can be executed only in the case where a treatment tool, such as forceps, has been captured in the observation image F and a detection value is thus able to be calculated more adequately.

### Third Modified Example

In the above described embodiment, the processor 412 determines, as the local areas Ar0, areas resulting from division of the observation image F (detection area) into 100 areas of 10×10, but the embodiment is not limited to this example.

FIG. 8 is a diagram for description of the third modified example of the embodiment. Specifically, FIG. 8 is a diagram illustrating the observation image F.

For example, as illustrated in FIG. 8, the processor 412 may divide the observation image F (detection area) into plural radial local areas Ar0 around a center position C of the observation image F.

In the case where the above described configuration according to the third modified example is adopted also, effects similar to those of the embodiment described above are achieved.

### Fourth Modified Example

In the above described embodiment, the reflection rate information is recorded in the memory 413 beforehand but the embodiment is not limited to this example. In a configuration that may be adopted instead, the reflection rate information is recorded as information specific to the endoscope 2 in the memory 241 of the endoscope 2 and the reflection rate information may be used by the processor 412 at Step S7.

In the case where the above described configuration according to the fourth modified example is adopted also, effects similar to those of the embodiment described above are achieved.

### Fifth Modified Example

In a configuration that may be adopted in the above described embodiment, Step S7 is executed only in the following case (and Step S8 is executed after Step S6 in a case where it is determined that it has not been captured therein).

Information indicating whether or not Step S7 is to be executed has been recorded in the memory 241 of the endoscope 2. The processor 412 obtains the information, and executes Step S7 in a case where the information is information indicating that Step S7 is to be executed and does not execute Step S7 in a case where the information is information indicating that Step S7 is not to be executed.

In the case where the above described configuration according to the fifth modified example is adopted also, effects similar to those of the embodiment described above are achieved.

A detection value calculation device, an endoscope system, a detection value calculation method, and a detection value calculation program, according to the disclosure enable adequate calculation of a detection value for adjusting brightness of an observation image.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A processing device comprising:
one or more processors comprising hardware, the one or more processors being configured to
receive an observation image captured by an endoscope,
calculate a representative luminance value of each of plural luminance areas resulting from classification of a specific detection area in an observation image according to luminance values,
determine a treatment tool area in the observation image,
count detected pixels exceeding a specific luminance value from pixels in the detection area,
reduce a counted number of the detected pixels in the treatment tool area from a counted number of the detected pixels of the pixels in the detection area,
calculate a weight of each of the plural luminance areas based on the counted number of the detected pixels subjected to the reducing, and
adjusting brightness of the observation image based on the weight and the representative luminance value of each of the plural luminance areas.

2. The processing device according to claim 1, wherein the one or more processors being configured to classify the specific detection area into the plural luminance areas that are a first area having a highest luminance value, a third area having a lowest luminance value, and one or more second areas having a luminance value intermediate between the highest luminance value of the first area and the lowest luminance value of the third area.

3. The processing device according to claim 1, wherein the one or more processors being configured to classify the specific detection area into the plural luminance areas that are an area of a first group of the plural luminance areas having high luminance values, an area of a third group of the plural luminance areas having low luminance values, and an area of a second group of the plural luminance areas having intermediate luminance values between the high luminance values of the first group and the low luminance values of the third group.

4. The processing device according to claim 2, wherein the one or more processors being configured to calculate representative luminance values of only the first area and the second area of the plural luminance areas.

5. The processing device according to claim 1, wherein the one or more processors being configured to divide the detection area into plural rectangular local areas and thereafter classify the plural local areas into the plural luminance areas.

6. The processing device according to claim 1, wherein the one or more processors being configured to divide the detection area into plural radial local areas around a center position of the observation image and thereafter classify the plural local areas into the plural luminance areas.

7. The processing device according to claim 1, wherein the one or more processors being configured to
receive treatment tool area information, from the endoscope, indicating the treatment area, and
determine the treatment area based on the treatment tool area information.

8. The processing device according to claim 1, wherein the one or more processors being configured to
determine whether or not a treatment tool has been captured in the observation image by performing image analysis of the observation image, and
execute the reducing when it is determined that the treatment tool has been captured in the observation image.

9. The processing device according to claim 8, wherein the one or more processors being configured to determine whether or not the treatment tool has been captured in the observation image by pattern matching.

10. The processing device according to claim 8, wherein the one or more processors being configured to determine whether or not the treatment tool has been captured in the observation image based on a luminance distribution of a specific area in the observation image.

11. An endoscope system, comprising:
the processing device according to claim 1;
a light source configured to illuminate a subject;
an imaging device configured to capture a subject image;
a processing device comprising one or more processors comprising hardware.

12. A processing method comprising:
receiving an observation image captured by an endoscope,
calculating a representative luminance value of each of plural luminance areas resulting from classification of a specific detection area in an observation image according to luminance values,
determining a treatment tool area in the observation image,
counting detected pixels exceeding a specific luminance value from pixels in the detection area,
executing adjustment processing of reducing a counted number of the detected pixels in the treatment tool area from a counted number of the detected pixels of the pixels in the detection area,
calculating a weight of each of the plural luminance areas based on the counted number of the detected pixels subjected to the reducing, and
adjusting brightness of the observation image based on the weight and the representative luminance value of each of the plural luminance areas.

13. The processing method according to claim 12, further comprising classifying the specific detection area into the plural luminance areas that are a first area having a highest luminance value, a third area having a lowest luminance value, and a one or more second areas having a luminance value that is intermediate between the highest luminance value of the first area and the lowest luminance value of the third area.

14. The processing method according to claim 12, further comprising classifying the specific detection area into the plural luminance areas that are an area of a first group of the plural luminance areas having a high luminance values, an area of a third group of the plural luminance areas having a low luminance values, and an area of a second group of the plural luminance areas having an intermediate luminance values between the high luminance values of the first group and the low luminance values of the third group.

15. The processing method according to claim 13, further comprising calculating representative luminance values of only the first area and the second area of the plural luminance areas.

16. The processing method according to claim 12, further comprising dividing the detection area into plural rectangular local areas and thereafter classify the plural local areas into the plural luminance areas.

17. The processing method according to claim 12, further comprising:
receiving treatment tool area information, from the endoscope, indicating the treatment area, and
determining the treatment area based on the treatment tool area information.

18. The processing method according to claim 12, further comprising:
determining whether or not a treatment tool has been captured in the observation image by performing image analysis of the observation image, and
executing the reducing when it is determined that the treatment tool has been captured in the observation image.

19. The processing method according to claim 18, further comprising determining whether or not the treatment tool has been captured in the observation image by pattern matching.

20. A non-transitory computer-readable recording medium with an executable program stored thereon, the program causing a processor to execute:
receiving an observation image captured by an endoscope,
calculating a representative luminance value of each of plural luminance areas resulting from classification of a specific detection area in an observation image according to luminance values,
determining a treatment tool area in the observation image,
counting detected pixels exceeding a specific luminance value from pixels in the detection area,
reducing a counted number of the detected pixels in the treatment tool area from a counted number of the detected pixels of the pixels in the detection area,
calculating a weight of each of the plural luminance areas based on the counted number of the detected pixels that has been subjected to the adjustment processing, and
adjusting brightness of the observation image based on the weight and the representative luminance value of each of the plural luminance areas.
